(19) United States
(12) Patent Application Publication
(10) Pub. No.: US 2018/0306763 A1
(43) Pub. Date: Oct. 25, 2018

(54) END POINT DETECTION FOR LYOPHILIZATION

(71) Applicant: MKS Instruments, Inc., Anodver, MA (US)

(72) Inventors: Gerardo A. Brucker, Longmont, CO (US); David W. Kelly, Johnstown, CO (US); Philip D. Acomb, San Ramon, CA (US)

(21) Appl. No.: 15/952,482

(22) Filed: Apr. 13, 2018

Related U.S. Application Data
(60) Provisional application No. 62/488,160, filed on Apr. 21, 2017.

(51) Int. Cl. G01N 33/15

(52) U.S. Cl. CPC G01N 33/15

(57) ABSTRACT

Methods and systems for endpoint detection of lyophilization processes are provided. A method for detecting an endpoint in a lyophilization process includes monitoring a total pressure of gases within a chamber containing a sample undergoing lyophilization and controlling a mass rate of flow of inert gas delivered to the chamber to replace water vapor removed from the chamber. The method further includes determining that sufficient water has been removed from the chamber based on total pressure and mass flow rate of inert gas being delivered.

### RELATED APPLICATION

This application claims the benefit of U.S. Provisional Application No. 62/488,160, filed on Apr. 21, 2017. The entire teachings of the above application are incorporated herein by reference.

### BACKGROUND

Lyophilization is an expensive and lengthy process used throughout the pharmaceutical industry to freeze dry labile chemicals. Lyophilization, also referred to as freeze drying, is the removal of water or other solvents from a product by sequential freezing (Thermal Treatment), vacuum sublimation (Primary Drying), and vacuum desorption (Secondary Drying). Lyophilization can provide products having shelf lives that significantly exceed those of air dried product. Most lyophilization systems operate without sensors to provide water content measurements during operation. As a result, primary and secondary drying times within a lyophilization process are selected during process development and are not adjusted on a process-by-process basis. Such fixed drying times can result in product that is not completely dried or, alternatively, in wasted time during production due to over-drying. As part of Process Analytical Technology (PAT) initiatives under development in the pharmaceutical industry, endpoint detection methodologies for primary and secondary drying processes are being included in lyophilization systems.

### SUMMARY OF THE INVENTION

Methods and systems for endpoint detection of lyophilization processes are provided. A method for detecting an endpoint in a lyophilization process includes monitoring a total pressure of gases within a chamber containing a sample undergoing lyophilization and controlling a mass rate of flow of inert gas delivered to the chamber to replace water vapor removed from the chamber. The method further includes determining that sufficient water has been removed from the chamber based on total pressure and mass flow rate of inert gas being delivered.

A method of detecting water content during a lyophilization process includes monitoring a total pressure of gases within a chamber containing a sample undergoing lyophilization and controlling a mass rate of flow of inert gas delivered to the chamber to replace water vapor removed from the chamber. The method further includes determining a water content in the chamber based on total pressure and mass flow rate of inert gas being delivered.

A lyophilization process includes monitoring a total pressure of gases within a chamber containing a sample undergoing lyophilization, removing water vapor from the chamber with a water pump, and controlling a mass rate of flow of inert gas delivered to the chamber to replace water removed from the chamber. The method further includes pumping inert gas from the chamber with a vacuum pump and determining that sufficient water has been removed from the chamber based on total pressure and mass flow rate of inert gas being delivered. When sufficient water has been removed, the lyophilization process is ended.

A system for detecting an endpoint in a lyophilization process includes a sensor that monitors a total pressure of gases within a chamber containing a sample undergoing lyophilization and a mass flow controller that controls a mass rate of flow of inert gas delivered to the chamber to replace water vapor removed from the chamber. The system further includes a controller configured to determine that sufficient water has been removed from the chamber based on total pressure and mass flow rate of inert gas being delivered.

A system for detecting water content during a lyophilization process includes a sensor that monitors a total pressure of gases within a chamber containing a sample undergoing lyophilization and a mass flow controller that controls a mass rate of flow of inert gas delivered to the chamber to replace water vapor removed from the chamber. The system further includes a controller configured to determine a water content in the chamber based on total pressure and mass flow rate of inert gas being delivered.

A lyophilization system includes a sensor that monitors a total pressure of gases within a chamber containing a sample undergoing lyophilization, a water pump that removes water vapor from the chamber, and a mass flow controller that controls a mass rate of flow of inert gas delivered to the chamber to replace water removed from the chamber. The system further includes a vacuum pump that pumps inert gas from the chamber and a controller configured to determine that sufficient water has been removed from the chamber based on total pressure and mass flow rate of inert gas being delivered. When sufficient water has been removed, the controller ends the lyophilization process.

The determination that sufficient water has been removed can include determining a partial pressure of water vapor in the chamber, which can fall beneath a threshold value for either a primary or secondary drying process. A partial pressure P_{H2O} of water vapor in the chamber can be determined, such as by a controller, according to the following:

$${P}_{H 2 O} = {P}_{T} - \frac{Q}{S} \qquad ( 1 )$$

where P_{T} is the total pressure, Q is the mass rate of flow of inert gas delivered to the chamber, and S is a volume rate of flow of inert gas being removed from the chamber. To determine the volume rate of flow S of inert gas being removed from the chamber, inert gas can be supplied to the chamber (e.g., while the chamber is empty, prior to lyophilization) and the volume rate of flow S can be determined, such as by a controller, according to the following:

$$S = \frac{{Q}_{R}}{{P}_{R}} \qquad ( 2 )$$

where P_{R} is a reference pressure and Q_{R} is a mass rate of flow of inert gas delivered to the chamber at the reference pressure. The inert gas can be a non-condensable gas, such that it passes through a water pump unaffected.

Alternatively, or in addition, the determination that sufficient water has been removed can include determining a change in the mass flow rate of inert gas being delivered to the chamber, which can fall beneath a threshold value for either a primary or secondary drying process.

During lyophilization, the volume rate of flow of inert gas being removed from the chamber can be maintained at a constant value by the vacuum pump. The lyophilization process can be a constant pressure process, in which the total pressure of gases within the chamber is maintained at a constant value.

The total pressure of gases within the chamber can be monitored by a capacitance manometer, which is species independent in providing total pressure. The total pressure, a percentage of the total pressure due to water vapor, a percentage of the total pressure due to inert gas, and/or the mass flow rate of the inert gas delivered to the chamber can be displayed.

### BRIEF DESCRIPTION OF THE DRAWINGS

The foregoing will be apparent from the following more particular description of example embodiments of the invention, as illustrated in the accompanying drawings in which like reference characters refer to the same parts throughout the different views. The drawings are not necessarily to scale, emphasis instead being placed upon illustrating embodiments of the present invention.

FIG. 1 is a graph illustrating a prior art lyophilization process.

FIG. 2 is a graph illustrating a prior art approach to detecting an endpoint of a primary drying process.

FIG. 3 is a schematic illustrating a system for the detection of an endpoint in a lyophilization process.

FIG. 4A is a simplified diagram illustrating partial pressures of gases during a lyophilization process.

FIG. 4B is a graph illustrating partial pressures of gases during primary and secondary drying phases of a lyophilization process.

### DETAILED DESCRIPTION OF THE INVENTION

A description of example embodiments of the invention follows.

Methods and systems for endpoint detection of lyophilization processes are provided that utilize pressure sensors and mass flow controllers regularly included in lyophilization systems. Such methods and systems can be used for both primary and secondary drying processes.

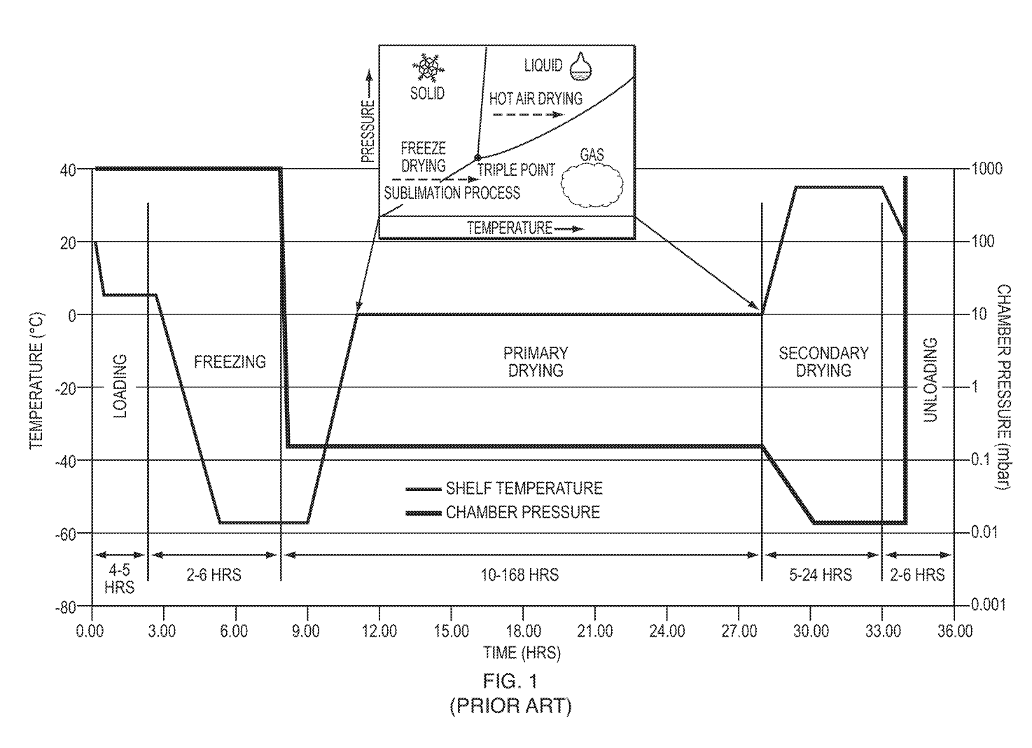

A typical lyophilization process is illustrated in FIG. 1. During an initial loading process, samples are placed in a lyophilization chamber. The samples are typically vials, flasks, or trays that contain a drug product (e.g., proteins, microbes, pharmaceuticals, tissues, or plasmas). The samples are then frozen in a process that can take about 2 to about 6 hours. Following the initial loading and freezing steps, the drying processes begin. During a primary drying process, frozen water (and other solvents) are removed from the product through sublimation. Sublimation is the process of changing from a solid to a gas without passing through an intermediate liquid phase. As shown in FIG. 1, sublimation occurs at pressures and temperatures that are below the triple point of water. To initiate the primary drying process, a vacuum is applied to the lyophilization chamber, causing pressure within the chamber to drop, and heat energy is added, causing the product to sublime. The sublimation process can take about 10 to about 168 hours, depending upon the size of the chamber, the number of samples contained in the chamber, and the water content of the samples. A majority of the water content of the samples is removed during the primary drying process.

A secondary drying process follows in which bound water molecules are removed by desorption. As shown in FIG. 1, during the secondary drying process, pressure within the chamber is again lowered, while additional heat is applied, causing bound water molecules to be released from the product. Because free ice within the sample has been removed during the primary drying process, the temperature may be increased during the secondary drying process without causing the product to melt or collapse. The desorption process can take about 5 to about 24 hours. After both drying processes have completed, the samples are unloaded from the chamber.

An important consideration in lyophilization processes is the determination of endpoints for both the primary and secondary drying stages. Moisture content can be in the range of, for example, about 5% to about 10%, at the end of a primary drying process and in the range of about 0.5% to about 3% at the end of a secondary drying process. The application of additional heat too early in a lyophilization process (e.g., before sublimation has completed) can cause melting or collapse of the product (often referred to as “cake collapse”). However, cost considerations make it undesirable to unnecessarily extend the time of a primary drying process. Also, various drugs may have different thresholds for acceptable residual moisture content. Generally, a longer shelf-life can be achieved by removing more moisture. However, some biological products can be over-dried if moisture content is brought below an acceptable threshold.

One methodology for detecting the endpoints of the drying processes involves the measurement of sample vial temperatures with thermocouples, such as wired or wireless thermocouples, during the drying process. An increase in sample temperature is expected when the frozen water is removed because the heat applied to the sample for sublimation is no longer being removed by vaporization of water. However, such an approach has a main disadvantage in that thermocouples that contact the sample can affect the nucleation of the product in the vial, providing a false indication of completion of the drying process, (i.e., not a bulk measurement).

Another approach involves the measurement of water content in the chamber during the drying process. Methodologies for this approach include the use of additional sensors that are capable of detecting water in the system, such as Pirani gauges, plasma emitters, and residual gas analyzers. One method, in particular, involves the use of a combination of capacitance diaphragm gauges and Pirani gauges to measure water content during primary and second drying processes. This methodology, further described in Patel, Sajal M., Takayuki Doen, and Michael J. Pikal. “Determination of End Point of Primary Drying in Freeze-Drying Process Control.” AAPS PharmSciTech 11.1 (2010): 73-84, has been found to minimize wasted time during lengthy primary drying processes. This methodology is often called Comparative Pressure Measurement (CPM) and is suited to lyophilization processes in which the total pressure in the system is kept constant. This method is gaining traction in the industry.

In constant pressure lyophilization processes, pressure is monitored with a capacitance diaphragm gauge and, as water vapor pressure drops during the drying process, an inert gas such as nitrogen is introduced into the system as needed to maintain a constant total pressure. Constant pressure lyophilization provides a continuous rate of heat exchange between the sample vials contained in the lyophilization chamber and the gas phase, providing for faster drying process cycles, particularly for primary drying processes.

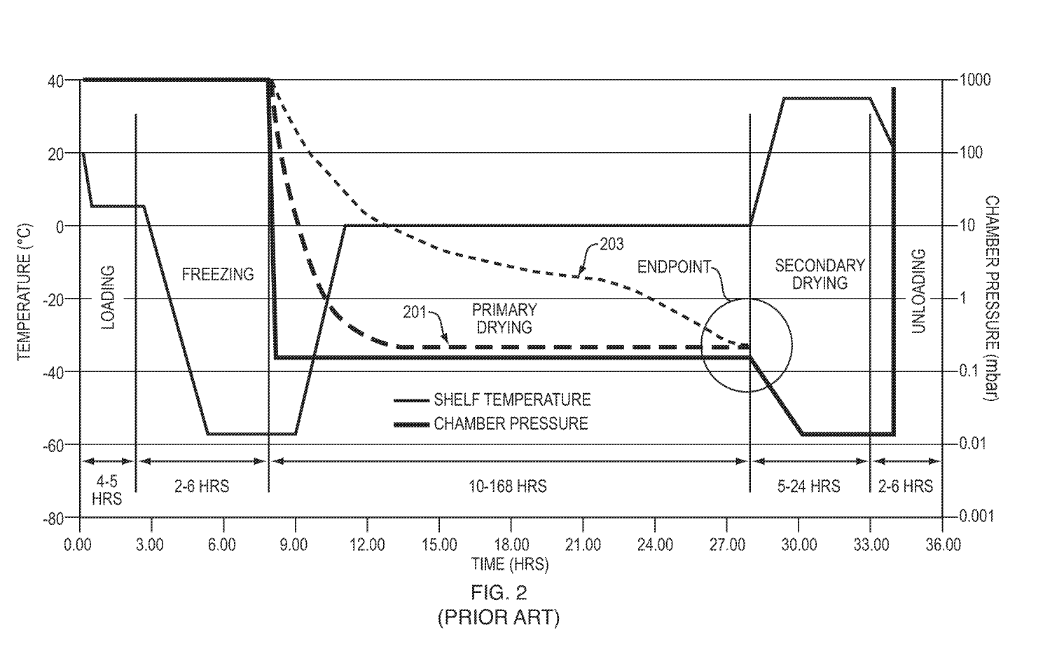

The pressure responses of a Pirani gauge and a capacitance diaphragm gauge during a constant pressure lyophilization process are shown in FIG. 2, where the species independent manometer gauge output 201 and the water vapor sensitive Pirani gauge output 203 are superimposed over the process diagram of FIG. 1. As shown in FIG. 2, the Pirani gauge readings initially overestimate the total pressure while the gas composition is dominated by water but eventually match the readings of the capacitance diaphragm gauge as water is removed from the chamber and the gas composition becomes dominated by nitrogen. Thus, towards the end of a drying process, a comparative measurement between the Pirani gauge output and the capacitance diaphragm gauge output can be taken to determine when sufficient water has been removed from the sample. One of the main advantages to CPM techniques is that Pirani gauges are not destructive to samples, unlike thermocouples, and bulk humidity measurements can be provided.

However the use of Pirani gauges in lyophilization processes faces several challenges. First, not all commercially available Pirani gauges are compatible with the clean-in-place (CIP) and/or sterilize-in-place (SIP) processes for lyophilization systems. Most Pirani gauges were not designed to provide adequate drainage after CIP and SIP processes, and even those Pirani gauges capable of withstanding such processes do not exhibit long times between failures. Second, Pirani gauges are not as accurate as capacitance diaphragm gauges (CDGs) and, as a result, present challenges for metrology labs in pharmaceutical industries that require measurement accuracies matching those provided by CDGs. Additionally, metrology labs are not well versed in calibration procedures for Pirani gauges and often do not have adequate experience to determine how often such gauges need to be calibrated or when such gauges show signs of inaccuracy. Third, Pirani gauges often provide inadequate output signals (e.g., S-curves), which creates difficulties for system integrators in the pharmaceutical industry to incorporate the gauges into the data acquisition systems of these tools. As such, Pirani gauges are perceived to have several shortcomings, and there is reluctance in the industry to include such low accuracy and unstable sensors into industrial processes. The majority of CPM systems are relegated to research and development (R&D) systems.

Methods and systems for endpoint detection of lyophilization processes are provided that obviate the need for Pirani sensors and that utilize equipment regularly included in lyopholization systems. Such methods can be applied to existing lyophilization systems without requiring any changes to system infrastructure other than the addition of a new controller for sensor integration. In particular, measurements of water content within a lyophilization chamber are performed based on a mass rate of flow from a mass flow controller (MFC) and a total pressure from a CDG, or any species independent pressure gauge, such as a piezoresistive diaphragm, a stress gauge, etc. CDGs are standard in lyophilization systems. MFCs are regularly included in constant pressure lyophilization systems to deliver an inert gas, such as nitrogen, into the lyophilization chamber to keep a total pressure within the chamber constant throughout a primary and/or secondary drying process.

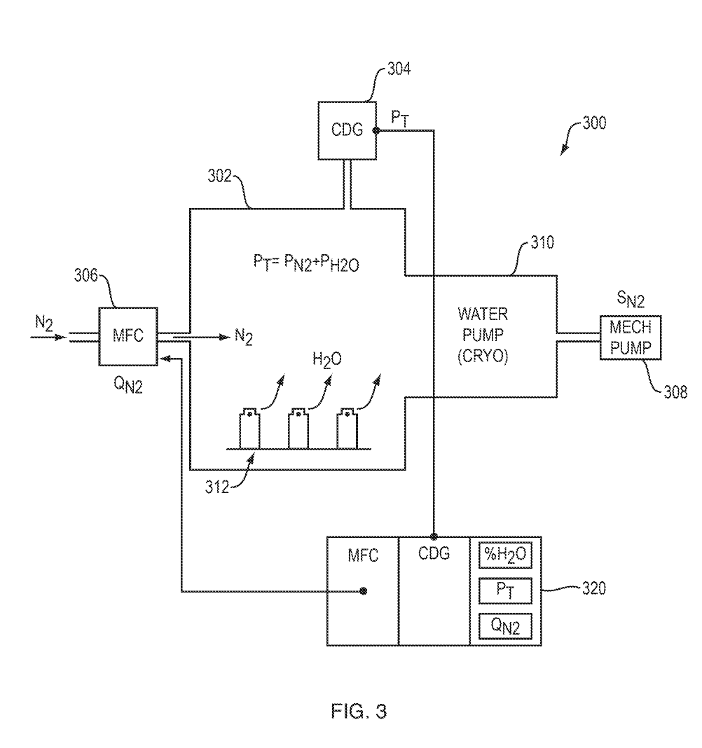

An example of a lyophilization system 300 is shown in FIG. 3. Connected to a lyophilization chamber 302 are a CDG 304, which is used to measure and control a total pressure (P_{T}) during a drying process, and an MFC 306, which is used to deliver a pure inert gas into the chamber as water content drops and as needed to keep a total pressure within chamber 302 constant. A pumping system, also connected to the chamber 302, includes a mechanical pump 308 and a water pump 310. The water pump 310 captures water during the drying process of samples 312. The water pump 310 can be a cryogenic pump, which pumps water out of the chamber at high pumping speeds via cryogenic capture and which does not capture the inert gas, such as nitrogen. The mechanical pump 308 is responsible for capturing non-condensable gases, which pass through the cryogenic pump unaffected. The mechanical pump 308 pumps the inert gas and other non-condensable gases out of the chamber 302.

Parameters of interest in the system 300 are a total pressure (P_{T}), a partial pressure of inert gas, such as nitrogen (P_{N2}), and a partial pressure of water (P_{H2O}). These parameters are related by the following:

P_{T}=P_{H2O}+P_{N2}. (3)

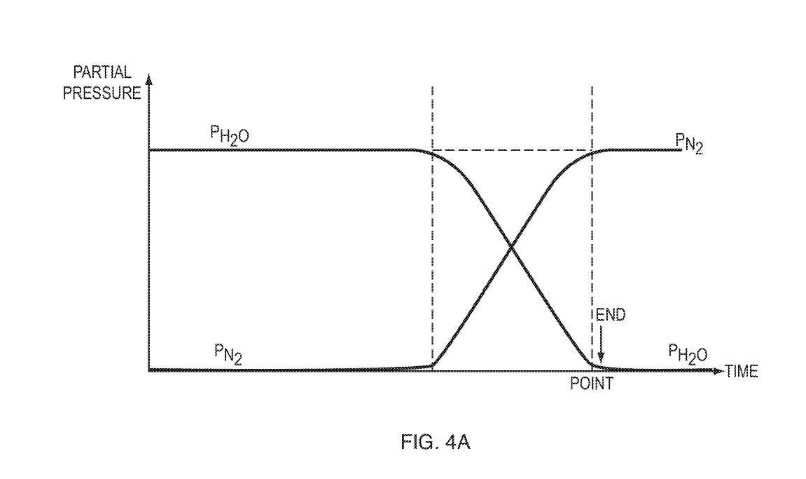

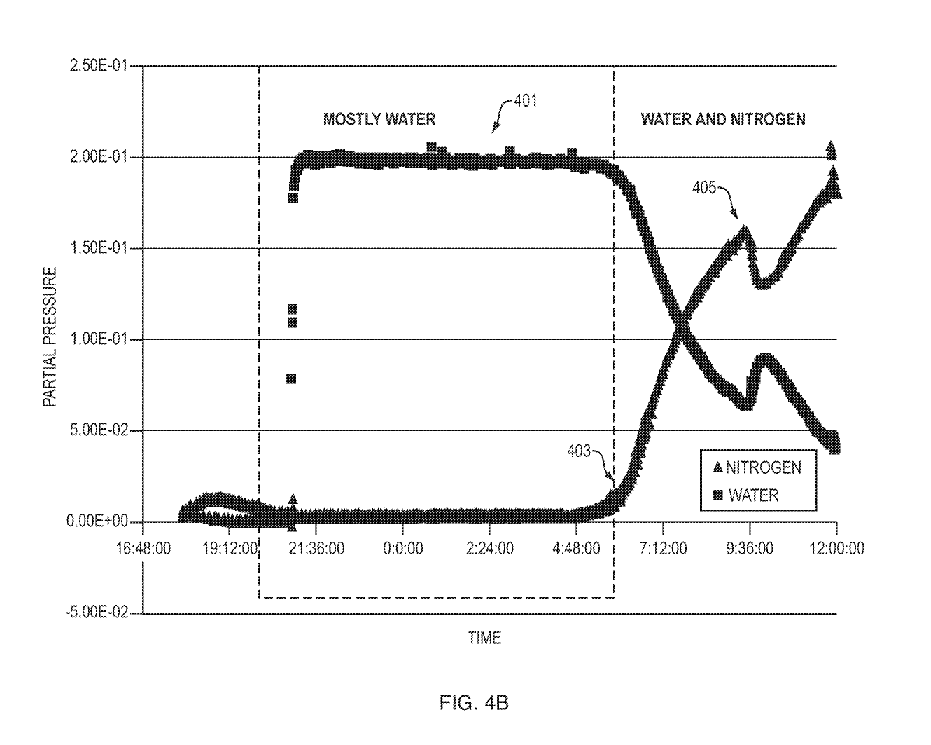

A simplified diagram illustrating the partial pressures of water and nitrogen in a constant pressure lyophilization system is shown in FIG. 4A. As shown in FIG. 4A and with regard to Eqn. 3, at any time during a lyophilization process, a total pressure within the system is equal to the partial pressures of water and nitrogen, with the total pressure P_{T} approximately equaling P_{H2O} near the beginning of the process and, once a majority of the water content of the sample has been removed, approximately equaling P_{N2} near the end of the process.

A more detailed diagram illustrating the partial pressures of water and nitrogen throughout both phases of a lyophization process are shown in FIG. 4B. During the primary drying process, the initial gas composition in the chamber is mostly water vapor, as shown at 401 in FIG. 4B. As the drying process progresses, water levels drop, and, in a constant pressure system, a mass flow controller introduces sufficient nitrogen flow such that nitrogen levels increase as needed to keep the total pressure level within the chamber constant, as shown at 403. The partial pressure of water vapor is expected to drop rapidly towards the end of a primary drying process. During the secondary drying process, the initial gas composition in the chamber is mostly nitrogen, with the partial pressure of water initially rising as heat is applied to the samples, as shown at 405, followed by another decline and essentially approaching zero at the end point. To determine endpoints for both primary and secondary drying processes, it is desirable to accurately measure the water content in the system. However, as described above, the inclusion of additional sensors, such as Pirani gauges, has several disadvantages. Alternatively, water content in system 300 can be obtained by measuring a contribution of water partial pressure to the total pressure in the system, without the inclusion of additional sensors.

In both primary and secondary drying processes, the CDG 304 maintains the total pressure P_{T} in the system constant by prompting nitrogen gas (N₂) to be added by the MFC 306 as required when water levels drop. The CDG provides a species independent measurement for P_{T}. During a drying process, water vapor is emitted from samples 312, which is then removed from the chamber and captured by the water pump 310. The MFC 306 adds N₂ to the chamber, in response to readings from the CDG, to compensate for the loss of water pressure during the drying process. A mass rate of flow Q_{N2} of nitrogen into the system (e.g., in units of Torr·L/s, or Pa·m³/s) can be provided by the MFC. The nitrogen introduced into the chamber is removed by the mechanical pump 308. The pumping speed of the mechanical pump is referred to as a volume rate of flow S_{N2} (e.g., in units of L/s, or m³/s). Thus, the partial pressure of nitrogen P_{N2} in the chamber 302 can be provided by the following:

P_{N2}=Q_{N2}/S_{N2}. (4)

where Q_{N2} is the mass rate of flow of nitrogen entering the system and S_{N2} is the volume rate of flow of nitrogen exiting the system.

Accordingly, combining equations (3) and (4) and rearranging terms, a partial pressure of water can be measured at any time during the drying process, according to the following:

P_{H2O}=P_{T}−Q_{N2}/S_{N2} (5)

where P_{T} is measured by the CDG, Q_{N2} is measured by the MFC, and S_{N2} is a constant for the mechanical pump.

A mechanical pump can operate at a given speed, such that the volume rate of flow S_{N2} is maintained at a constant value throughout a drying process. The volume rate of flow S_{N2} for a mechanical pump can be determined by supplying pure nitrogen to an unloaded chamber that has been pumped down to base pressure and activating an MFC to deliver pure nitrogen until a reference pressure P_{R} is obtained, as measured by the CDG. Upon reaching the reference pressure, a reference mass rate of flow Q_{R} can be measured by the MFC. The volume rate of flow S_{N2} can then be calculated according to the following:

S_{N2}=Q_{R}/P_{R}, (6)

which can be used as a constant in Equations 4 and 5.

Returning to FIG. 3, the system 300 also includes a controller 320 connected to the CDG 304 and MFC 306. Controller 320 can be configured to accept a value for S_{N2}, if known, or to determine a value for S_{N2}. With a parameter for S_{N2} in memory, the controller 320 can calculate a partial pressure of water vapor in the system at any given time (by Eqn. 5). The controller can also be configured to calculate a percentage of water content % H2O in the system according to the following:

% H2O=P_{H2O}/P_{T}. (7)

where P_{H2O} is the partial pressure of water vapor in the system and P_{T} is the total pressure in the system.

Thus, the controller can provide an accurate measurement of water content in the system throughout a drying process, and can further display and/or record % H2O and P_{H2O}. In addition to displaying a measure of water content in the system, the controller can further display total pressure P_{T}, partial pressure of nitrogen P_{N2}, mass flow rate Q_{N2} of nitrogen, and/or volume flow rate S_{N2} of nitrogen. Threshold values for a partial pressure of water vapor or a percent of water vapor in the system can be preselected, with the controller providing an alert or automatically ending a drying process when threshold value(s) are reached.

Alternative to computing % H2O, the controller can monitor the mass flow rate Q_{N2} of nitrogen. As can be seen by Eqn. 4, the mass flow rate Q_{N2} during a lyophilization process follows the curve of the partial pressure P_{N2} of nitrogen (FIG. 4A) multiplied by a constant S_{N2}. The controller can be configured to obtain a derivative of a Q_{N2} curve to determine that nitrogen flow is stabilizing and that the amount of water vapor in the system is leveling off. Thresholds can be programmed for the end of primary and/or secondary drying processes based off of a derivative of the Q_{N2} curve to indicate endpoints. Alternatively, a derivative of the Q_{N2} a can be used in combination with % H2O or P_{H2O} to determine an endpoint. For example, if the water content is beneath a threshold and nitrogen flow is no longer changing at an adequate rate, the controller can determine that an endpoint has been reached.

The controller can also include a Proportional Integrated Derivative (PID) Control Loop to allow a user to control total pressure in the system by reading P_{T} and delivering a proper amount of N₂ to keep total pressure constant. As such, system 300, using an existing infrastructure of CDG sensors and MFCs, can control pressure throughout a lyophilization processes, monitor water content during both primary and secondary drying processes, and issue an endpoint signal in each of the primary and secondary drying processes as water levels drop to respective specified, threshold levels.

The measurement of water content based on total pressure readings from a CDG and mass flow rate readings of an MFC offers several advantages. In particular, the use of Pirani gauges is obviated. As described above, Pirani gauges present accuracy drift issues, sensitivity to CIP and SIP processes, and provide too much variability in performance between different vendors. Pirani gauges are not specifically designed for lyophilization. In contrast, CDGs are more accurate (e.g., <0.025% error) than Pirani gauges (e.g., 5% error) and are compatible with CIP and SIP processes. CDGs and MFCs are already vetted for lyophilization applications and are routinely used in such applications. CDGs and MFCs are compatible with modern Good Manufacturing Practices (GMP) of the pharmaceutical industry. As such, the methods described above do not require the use of any sensor or other equipment that is of unknown compatibility with lyophilization processes or that presents unknown accuracy drift issues.

Additionally, the methods and systems described above can handle several operations involved in lyophilization processes, including both pressure control and endpoint detection. Procedures, such as ending a process based on a threshold water content value having been reached, can be programmed into the system and controlled via digital logic or a command level interface.

Lastly, the systems described above can also provide system diagnostic data. For example, by performing a measurement of P_{T} and Q_{N2} between runs and by using pure nitrogen gas, a user can quickly diagnose that the sensors and pumps are operating properly. If any measured values for S_{N2}, Q_{N2}, and/or P_{N2} deviate from those values as obtained by an initial S_{N2} calculation, a fault report can be generated to investigate which component (e.g., mechanical pump, CDG, and/or MFC) has drifted away from its initial calibrated state. As such, the system includes a built-in diagnostic that enables a user to perform a system check prior to each run.

As lyophilization systems often already include CDGs, systems as described above can be retrofitted into existing tools that do not utilize constant pressure control through the addition of an MFC and a controller configured to receive measurements from the CDG, operate the MFC, and perform the above-described methods. Such a controller can also be added to existing constant pressure setups to perform the methods described above. Alternatively, existing controllers can be reprogrammed to perform the described methods. The added or reprogrammed controllers can also include diagnostics of the MFC, CDG, and pumps, which can be used between runs to verify that equipment is operating normally.

Optionally, a Pirani gauge can be included in system 300 to provide a redundant measurement of water content, such as by a gauge comparison method. If a Pirani gauge is included, it can be recalibrated at the beginning of each drying process by comparing its readings to those of a CDG while the chamber is empty of samples and filled with pure nitrogen.

While system 300 and Eqns. 3-5 have been described with regard to nitrogen being the inert gas that is provided to the system, it should be understood that any inert gas that can be used in a lyophilization process can be used in systems and methods of the present invention. Nitrogen is often used in lyophilization processes because it is inexpensive and inert. Also, most Pirani gauges are factory calibrated against nitrogen. In addition to being inert, nitrogen does not condense in a cryopump. In the methods and systems described above, the use of nitrogen, which is non-condensable, provides for easily determining the pumping speed of a mechanical pump and obtaining a value for SN2. However, other non-condensable gases can also be used.

The teachings of all patents, published applications and references cited herein are incorporated by reference in their entirety.

While this invention has been particularly shown and described with references to example embodiments thereof, it will be understood by those skilled in the art that various changes in form and details may be made therein without departing from the scope of the invention encompassed by the appended claims.

What is claimed is:

1. A method of determining an endpoint in a lyophilization process, comprising:
monitoring a total pressure of gases within a chamber containing a sample undergoing lyophilization;
controlling a mass rate of flow of inert gas delivered to the chamber to replace water vapor removed from the chamber; and
determining that sufficient water has been removed from the chamber based on total pressure and mass flow rate of inert gas being delivered.

2. The method of claim 1, wherein determining that sufficient water has been removed from the chamber comprises determining a change in the mass flow rate of inert gas being delivered to the chamber and that the change has fallen beneath a threshold value.

3. The method of claim 1, wherein determining that sufficient water has been removed from the chamber comprises determining a partial pressure P_{H2O} of water vapor in the chamber and that the partial pressure P_{H2O} of water vapor in the chamber has fallen beneath a threshold value.

4. The method of claim 3, further comprising determining a partial pressure P_{H2O} of water vapor in the chamber according to the following:
$${P}_{H 2 O} = {P}_{T} - \frac{Q}{S}$$
where P_{T} is the total pressure, Q is the mass rate of flow of inert gas delivered to the chamber, and S is a volume rate of flow of inert gas being removed from the chamber.

5. The method of claim 4 further comprising:
supplying inert gas to the chamber; and
determining the volume rate of flow S of inert gas being removed from the chamber according to the following:
$$S = \frac{{Q}_{R}}{{P}_{R}}$$
where P_{R} is a reference pressure and Q_{R} is a mass rate of flow of inert gas delivered to the chamber at the reference pressure.

6. The method of claim 1, further comprising maintaining a volume rate of flow of inert gas being removed from the chamber at a constant value during lyophilization.

7. The method of claim 1, further comprising controlling the mass rate of flow of inert gas delivered to the chamber to maintain the total pressure of gases within the chamber at a constant value during lyophilization.

8. The method of claim 1, wherein the inert gas is non-condensable.

9. A system for determining an endpoint in a lyophilization process, comprising:
a sensor that monitors a total pressure of gases within a chamber containing a sample undergoing lyophilization;
a mass flow controller that controls a mass rate of flow of inert gas delivered to the chamber to replace water vapor removed from the chamber; and
a controller configured to determine that sufficient water has been removed from the chamber based on total pressure and mass flow rate of inert gas being delivered.

10. The system of claim 9, wherein the controller is further configured to determine a change in the mass flow rate of inert gas being delivered to the chamber and that the change has fallen beneath a threshold value for sufficient water having been removed from the chamber.

11. The system of claim 9, wherein the controller is further configured to determine a partial pressure P_{H2O} of water vapor in the chamber and that the partial pressure P_{H2O} of water vapor in the chamber has fallen beneath a threshold value for sufficient water having been removed from the chamber.

12. The system of claim 11, wherein the controller is further configured to calculate a partial pressure P_{H2O} of water vapor in the chamber according to the following:
$${P}_{H 2 O} = {P}_{T} - \frac{Q}{S}$$
where P_{T} is the total pressure, Q is the mass rate of flow of inert gas delivered to the chamber, and S is a volume rate of flow of inert gas being removed from the chamber.

13. The system of claim 12, wherein the controller is further configured to:
supply inert gas to the chamber; and
calculate the volume rate of flow S of inert gas being removed from the chamber according to the following:
$$S = \frac{{Q}_{R}}{{P}_{R}}$$
where P_{R} is a reference pressure and Q_{R} is a mass rate of flow of inert gas delivered to the chamber at the reference pressure.

14. The system of claim 9, wherein the sensor is a capacitance diaphragm gauge.

15. The system of claim 9, wherein the vacuum pump maintains a volume rate of flow of inert gas being removed from the chamber at a constant value during lyophilization.

16. The system of claim 9, wherein the controller is further configured to control the mass rate of flow of inert gas delivered to the chamber to maintain the total pressure of gases within the chamber at a constant value during lyophilization.

17. The system of claim 9, wherein the inert gas is non-condensable.

18. The system of claim 9, wherein the controller is further configured to display a percentage of the total pressure due to water vapor.

19. The system of claim 9 wherein the controller is further configured to display the total pressure and the mass flow rate of inert gas delivered to the chamber.

20. A method of detecting water content during a lyophilization process, comprising:
monitoring a total pressure of gases within a chamber containing a sample undergoing lyophilization;
controlling a mass rate of flow of inert gas delivered to the chamber to replace water vapor removed from the chamber; and
determining a water content in the chamber based on total pressure and mass flow rate of inert gas being delivered.

21. A lyophilization process, comprising:
monitoring a total pressure of gases within a chamber containing a sample undergoing lyophilization;
removing water vapor from the chamber with a water pump;
controlling a mass rate of flow of inert gas delivered to the chamber to replace water removed from the chamber;
pumping inert gas from the chamber with a vacuum pump;
determining that sufficient water has been removed from the chamber based on total pressure and mass flow rate of inert gas being delivered; and
ending the lyophilization process when sufficient water has been removed.

22. A system for detecting water content during a lyophilization process, comprising:
a sensor that monitors a total pressure of gases within a chamber containing a sample undergoing lyophilization;
a mass flow controller that controls a mass rate of flow of inert gas delivered to the chamber to replace water vapor removed from the chamber; and
a controller configured to determine a water content in the chamber based on total pressure and mass flow rate of inert gas being delivered.

23. A lyophilization system, comprising:
a sensor that monitors a total pressure of gases within a chamber containing a sample undergoing lyophilization;
a water pump that removes water vapor from the chamber;
a mass flow controller that controls a mass rate of flow of inert gas delivered to the chamber to replace water vapor removed from the chamber;
a vacuum pump that pumps inert gas from the chamber; and
a controller configured to:
determine that sufficient water has been removed from the chamber based on total pressure and mass flow rate of inert gas being delivered, and
end the lyophilization process when sufficient water has been removed.
